(19) United States
(12) Patent Application Publication
(10) Pub. No.: US 2008/0103202 A1
(43) Pub. Date: May 1, 2008

(54) METHOD OF PREPARING CREATINE ESTER SALTS AND USES THEREOF.

(75) Inventors: Chris Ferguson, Boca Raton, FL (US); Jiang Shengli, Shanghai (CN)

Correspondence Address: AMIN HALLIHAN, LLC, 217 N. JEFFERSON ST., SUITE 100, CHICAGO, IL, 60661, US

(73) Assignees: Chris Ferguson, Boca Raton, FL (US); Jiang Shengli, Shanghai (CN)

(21) Appl. No.: 10/904,389

(22) Filed: Nov. 8, 2004

(51) Int. Cl. A61K 31/22; C07C 279/10

(52) U.S. Cl. 514/551; 560/168

(57) ABSTRACT

This invention discloses the method of preparation of creatine ester-salts. Creatine is an extremely popular ergogenic aid, and is found most often in the form of creatine monohydrate. Creatine monohydrate is poorly soluble in water however and while esters gain solubility, there functionality is greatly decreased. The material can be administered in a variety of ways including capsules, tablets, powdered beverages, bars, gels, liquids, liposomes or drinks.

### FIELD OF THE INVENTION

The present invention discloses a method of preparing ester salts of creatine and methods of using creatine ester-salts to enhance creatine functionality and bioavailability for purposes of performance and lean mass enhancement, both in humans and animals.

### BACKGROUND

Creatine is the most popular performance enhancing supplement. Although creatine use has dated back to the early 1900's, its use was not commonplace until recent years. The fuel for all muscular work in the body is adenosine tri-phosphate, or ATP. During intense exercise, ATP is utilized very rapidly. The body does not store much ATP in muscle so other substances must be broken down in order to replenish the ATP that is rapidly broken down during exercise. If the ATP is not replenished, fatigue occurs and force/power production declines. Of all the substances in the body that can replenish ATP, the fastest is phosphorylated creatine. Thus, the primary function of phosphorylated creatine in muscle is to buffer ATP by preventing decreases in ATP during exercise and restoring ADP to its original tri-phosphate energy-producing form.

Creatine is taken up into tissues, such as skeletal muscle, by means of an active transport system that typical involves an insulin dependent pathway and sodium gradient. Typical levels of total creatine in skeletal muscle prior to administration are between about 100 to about 140 mmol/kg of dry muscle. The most common form of creatine used is Creatine monohydrate which has fairly poor solubility, particularly at a neutral pH and lower temperature fluids. Other forms of creatine have been introduced to the market such as micronized versions and other forms including magnesium bound, titrate, malate and many others.

U.S. Pat. No. 6,211,407 discloses a method of preparing a dicreatine citrate or tricreatine citrate, comprising two and three creatine cations per citrate anion, respectively.

Patent Application #20040077902 discloses Dicreatine maleate and methods of manufacturing a form of creatine which offers a level of water solubility more than 12 fold better than creatine monohydrate.

U.S. Pat. No. 6,166,249 discloses creatine pyruvates, for use to enhance long-term performance and strength in the field of sport, to reduce weight and body fat in the field of health, to treat conditions of oxygen deficit (ischemia), obesity and overweight, as food supplements and radical scavenger.

These forms all tout to offer various enhancements in functionality and bioavailability, but research is greatly lacking and there efficacy is questionable. As stated above, creatine is not particularly soluble, nor is it very well absorbed from the gastrointestinal tract. Thus, to achieve an effective dose, fairly large amounts of creatine are typically consumed, typically in excess of 10 grams per day, oftentimes 20 grams or more. In addition to the added expense, side effects are often seen with these higher doses and can cause side effects such as bloating and gastrointestinal distress.

To alleviate some of the original inherent flaws of creatine in recent years its use has been coupled with carbohydrates based upon research that suggested the insulin spike generated from the carbohydrates facilitated the transport of creatine into skeletal tissue. For example, in a study by Stengee et al., insulin was co-infused along with creatine supplementation. (Am. J. Physiol., 1998; 275:E974-79). The results of this study indicated that insulin can enhance creatine accumulation in muscle, but only if insulin levels are present at extremely high or supra-physiological concentrations. Stengee et al. refers to a previous study by Green et al. which involved experimentation with ingestion of creatine in combination with a carbohydrate-containing solution to increase muscular uptake of creatine by creating physiologically high plasma insulin concentrations. Stengee et al. reports that Green et al. had found the quantity of carbohydrate necessary to produce a significant increase in creatine uptake, as compared to creatine supplementation alone, was close to the limit of palatability. Theoretically, the more creatine that can be given at the moment of highest insulin concentration would promote the most rapid absorption of creatine into muscles and thus would provide maximum benefit to creatine users.

Also, in recent years combining creatine with various other insulin potentiating agents aside from carbohydrates has become quite common. Agents such as Pinitol, alpha-lipoic acid, 4-hydroxyisoleucine, taurine, arginine, chromium and many others have been used either in conjunction or in replacement of carbohydrates. Similar to the data above on carbohydrates and increased creatine deposition, it is often theorized that agents like these that purport to have effects on glucose control and insulin release can act to increase the absorption and deposition of creatine into the muscle cells.

In common practice in the pharmaceutical world today is the use of various esters and ethers, known agents to increase the solubility of chemicals. Recently a similar technology has been employed to the use of creatine due its known functional benefits of enhancing the solubility and potentially bioavailability of the compound. Therefore, compared with other forms of creatine, ethers and esters have greater solubility and permeability across the GI tract. Since, the more carbons the ester has, the lower the water solubility becomes and the higher the partition coefficient, the preferred ester for creatine is one with few carbons on the ester chain. Once in the GI tract, Creatine esters are converted by esterases in the intestine and blood to the biologically active form or unbound form of creatine. This applies however only to creatine that is delivered via a liquid. If the creatine is to be delivered via a softgel, liposomal or other oil based delivery system, the number of carbons should be much higher with a lower partition coefficient. From that point creatine can then be taken up and utilized by the muscle cells in typical fashion. In addition, creatine esters are resistant to the common conversion to creatinine in the acid environment of the stomach, another factor known to reduce bioavailability of creatine. Therefore, for maximum absorption and protection of the creatine molecule, esters or ethers of creatine should be utilized. However, one flaw with esters is that they have a particularly bad taste and therefore greatly lack in functionality.

Thus it is the object of this invention to disclose a method of preparing an ester-salt of creatine that 1) has increased bioavailability of creatine and 2) maintains and improves upon the functional nature and diversity of creatine by enhancing solubility and taste. Additionally, this invention will detail the methods of use of creatine-ester containing salts for purposes of performance and lean mass enhancement, both in humans and animals.

### SUMMARY

Disclosed herein is: (a) A method of preparing ester salts of creatine. (b) A dietary supplement comprising of creatine ester-salts and/or derivatives thereof, and (c) methods of increasing creatine functionality and bioavailability in mammalian muscle, and enhancing athletic performance and lean body mass comprising administration of said dietary supplement.

### BRIEF DESCRIPTION OF THE PREFERRED EMBODIMENT

Accordingly, it is an object of the invention to provide a method and a dietary supplement which will enhance the uptake of creatine into mammalian muscle. More specifically, it is an object of the invention to provide a method and a dietary supplement which will enhance the uptake of creatine into skeletal muscle whilst enhancing the functionality of the creatine molecule as well. It is a still further object of the invention to provide a method and a dietary supplement that achieves these objects when administered in physiologically acceptable amounts.

Other objectives, advantages and features of the invention will become apparent from the following detailed description, and from the claims.

### EXAMPLE 1

Under room temperature, 1000 mL water and 159 grams of creatine ethyl ester (1 mol) were added into a reaction flask under agitation, then, 67 grams of malic acid (0.5 mol) was added into the flask. The solution gradually converted from hazy to clean. When the solution is completely clean, remove most of the water by distillation under reduced pressure. The remaining mixture was chilled to 0 Celsius Degree below, filtered the mixture, Dicreatine Ethyl Ester Malate was obtained.

### EXAMPLE 2

Under room temperature, 159 gram of creatine ethyl ester (1 mol) and 67 grams of malic acid (0.5 mol) were added to 1000 mL Alcohol under agitation and stirring for 10 hours. Remove most of the alcohol by distillation under reduced pressure. The remaining mixture was chilled to less than 0 Celsius Degree, filtered the mixture, Dicreatine Ethyl Ester Malate was obtained.

Monocreatine esters malate can be similarly prepared.

### EXAMPLE 3

Under room temperature, 1000 mL water and 159 grams of creatine ethyl ester (1 mol) were added into a reaction flask under agitation, then, 58 grams of fumaric acid (0.5 mol) was added into the flask. The solution gradually converted from hazy to clean. When the solution is completely clean, remove most of the water by distillation under reduced pressure. The remaining mixture was chilled to 0 Celsius Degree below, filtered the mixture, Dicreatine Ethyl Ester Fumarate was obtained.

### EXAMPLE 4

Under room temperature, 159 gram of creatine ethyl ester (1 mol) and 58 grams of fumaric acid (0.5 mol) were added to 1000 mL Alcohol under agitation and stirring for 10 hours. Remove most of the alcohol by distillation under reduced pressure. The remaining mixture was chilled to less than 0 Celsius Degree, filtered the mixture, Dicreatine Ethyl Ester Fumarate was obtained.

Monocreatine esters fumarate can be similarly prepared.

### EXAMPLE 5

Under room temperature, 1000 mL water and 159 grams of creatine ethyl ester (1 mol) were added into a reaction flask under agitation, then, 88 grams of pyruvic acid (1 mol) was added into the flask. The solution gradually converted from hazy to clean. When the solution is completely clean, remove most of the water by distillation under reduced pressure. The remaining mixture was chilled to 0 Celsius Degree below, filtered the mixture, Creatine Ethyl Ester Pyruvate was obtained.

### EXAMPLE 6

Under room temperature, 159 gram of creatine ethyl ester (1 mol) and 88 grams of pyruvic acid (1 mol) were added to 1000 mL Alcohol under agitation and stirring for 10 hours. Remove most of the alcohol by distillation under reduced pressure. The remaining mixture was chilled to less than 0 Celsius Degree, filtered the mixture, Creatine Ethyl Ester Pyruvate was obtained.

### EXAMPLE 7

Under room temperature, 159 grams of creatine ethyl ester (1 mol) was mixed with 88 grams of pyruvic acid in a beaker. The mixture is left to stand, ultimately solidifying to a white, finely crystalline product. It was ground in a mortar and dried for 4 hours at 40-60 Clesius Degree. Creatine Ethyl Ester Pyruvate was obtained.

### EXAMPLE 8

Under room temperature, 159 gram of creatine ethyl ester (1 mol) and 73 grams of Alpha-ketoglutaric acid (0.5 mol) were added to 1000 mL Alcohol under agitation and stirring for 10 hours. Remove most of the alcohol by distillation under reduced pressure. The remaining mixture was chilled to less than 0 Celsius Degree, filtered the mixture, DiCreatine Ethyl Ester Alpha-ketoglutarate was obtained.

### EXAMPLE 9

Under room temperature, 1000 mL water and 159 grams of creatine ethyl ester (1 mol) were added into a reaction flask under agitation, then, 73 grams of Alpha-ketoglutaric acid (0.5 mol) was added into the flask. The solution gradually converted from hazy to clean. When the solution is completely clean, remove most of the water by distillation under reduced pressure. The remaining mixture was chilled to 0 Celsius Degree below, filtered the mixture, DiCreatine Ethyl Ester Alpha-ketoglutarate was obtained.

Monocreatine esters alpha-ketoglutarate can be similarly prepared.

### EXAMPLE 10

Under room temperature, 159 gram of creatine ethyl ester (1 mol) and 75 grams of Tartaric acid (0.5 mol) were added to 1000 mL Alcohol under agitation and stirring for 10 hours. Remove most of the alcohol by distillation under reduced pressure. The remaining mixture was chilled to less than 0 Celsius Degree, filtered the mixture, DiCreatine Ethyl Ester Tartrate was obtained.

### EXAMPLE 11

Under room temperature, 1000 mL water and 159 grams of creatine ethyl ester (1 mol) were added into a reaction flask under agitation, then, 75 grams of Tartaric acid (0.5 mol) was added into the flask. The solution gradually converted from hazy to clean. When the solution is completely clean, remove most of the water by distillation under reduced pressure. The remaining mixture was chilled to 0 Celsius Degree below, filtered the mixture, DiCreatine Ethyl Ester Tartrate was obtained.

Monocreatine esters Tartarte can be similarly prepared.

### EXAMPLE 12

Under room temperature, 159 gram of creatine ethyl ester (1 mol) and 64 grams of Citric acid (0.33 mol) were added to 1000 mL Alcohol under agitation and stirring for 10 hours. Remove most of the alcohol by distillation under reduced pressure. The remaining mixture was chilled to less than 0 Celsius Degree, filtered the mixture, TriCreatine Ethyl Ester Citrate was obtained.

### EXAMPLE 13

Under room temperature, 1000 mL water and 159 grams of creatine ethyl ester (1 mol) were added into a reaction flask under agitation, then, 64 grams of Tartaric acid (0.33 mol) was added into the flask. The solution gradually converted from hazy to clean. When the solution is completely clean, remove most of the water by distillation under reduced pressure. The remaining mixture was chilled to 0 Celsius Degree below, filtered the mixture, TriCreatine Ethyl Ester Citrate was obtained.

Monocreatine esters Citrate and Dicreatine esters citrate can be similarly prepared.

### EXAMPLE 14

Under room temperature, 1000 mL water and 159 grams of creatine ethyl ester (1 mol) were added into a reaction flask under agitation, then, 58 grams of Malic acid (0.5 mol) was added into the flask. The solution gradually converted from hazy to clean. When the solution is completely clean, remove most of the water by distillation under reduced pressure. The remaining mixture was chilled to 0 Celsius Degree below, filtered the mixture, Dicreatine Ethyl Ester Malate was obtained.

### EXAMPLE 15

Under room temperature, 159 gram of creatine ethyl ester (1 mol) and 58 grams of Maleic acid (0.5 mol) were added to 1000 mL Alcohol under agitation and stirring for 10 hours. Remove most of the alcohol by distillation under reduced pressure. The remaining mixture was chilled to less than 0 Celsius Degree, filtered the mixture, Dicreatine Ethyl Ester Malate was obtained.

Monocreatine esters maleate can be similarly prepared.

### EXAMPLE 16

Under room temperature, 1000 mL water and 159 grams of creatine ethyl ester (1 mol) were added into a reaction flask under agitation, then, 176 grams of Ascorbic acid (1 mol) was added into the flask. The solution gradually converted from hazy to clean. When the solution is completely clean, remove most of the water by distillation under reduced pressure. The remaining mixture was chilled to 0 Celsius Degree below, filtered the mixture, Creatine Ethyl Ester Ascorbate was obtained.

### EXAMPLE 17

Under room temperature, 159 gram of creatine ethyl ester (1 mol) and 176 grams of Ascorbic acid (1 mol) were added to 1000 mL Alcohol under agitation and stirring for 10 hours. Remove most of the alcohol by distillation under reduced pressure. The remaining mixture was chilled to less than 0 Celsius Degree, filtered the mixture, Creatine Ethyl Ester Ascorbate was obtained. Potential applications for creatine ester salts:

Formula 1

| | | |
|---|---|---|
| Dicreatine Ethyl Ester Malate | 2.0 | g |
| Alpha-lipoic acid | 100 | mg |
| Magnesium/Potassium Phosphate | 300 | mg |

Formula 2

| | | |
|---|---|---|
| Creatine Ethyl Ester Ascorbate | 1.5 | g |
| 4-hydroxylsoleucine | 200 | mg |
| L-Taurine | 500 | mg |
| D-Pinitol | 50 | mg |

Formula 3

| | | |
|---|---|---|
| Creatine Ethyl Ester Pyruvate | 1.5 | g |
| HMB | 1.0 | g |
| L-Taurine | 500 | mg |
| Cinnamon extract | 200 | mg |

Formula4

| | | |
|---|---|---|
| TriCreatine Ethyl Ester Citrate | 1.5 | g |
| Ruteacarpine | 50 | mg |
| Cinnamon extract | 200 | mg |
| L-Arginine AKG | 1.5 | g |

### Powdered Formulation

Formula 1

| | | |
|---|---|---|
| Dicreatine Ethyl Ester Malate | 5.0 | g |
| Alpha-lipoic acid | 100 | mg |
| Magnesium Phosphate | 300 | mg |
| Dextrose | 34.0 | g |
| L-Taurine | 1.0 | g |
| L-Glutamine | 2.0 | g |
| Di-potassium phosphate | 200 | mg |
| L-Arginine AKG | 2.0 | g |
| Rutacearpine | 50 | mg |

### Flavor and Sweetener to Taste

Formula 2

| | | |
|---|---|---|
| Creatine Ethyl Ester Ascorbate | 1.5 | g |
| 4-hydroxylsoleucine | 200 | mg |
| L-Taurine | 500 | mg |
| D-Pinitol | 50 | mg |
| L-Taurine | 1.0 | g |
| L-Glutamine | 2.0 | g |
| Di-potassium phosphate | 200 | mg |
| L-Arginine AKG | 2.0 | g |
| Cinnamon extract | 200 | mg |
| HMB | 1.0 | g |

### Flavor and Sweetener to Taste

Formula 3

| | | |
|---|---|---|
| Creatine Ethyl Ester Ascorbate | 1.5 | g |
| Flavor and Sweetener to taste | | |
| 4-hydroxylsoleucine | 200 | mg |
| L-Taurine | 500 | mg |
| D-Pinitol | 50 | mg |
| L-Taurine | 1.0 | g |
| Betaine HCL | 3.0 | g |
| L-Glutamine | 2.0 | g |
| Di-potassium phosphate | 200 | mg |
| TriCreatine Ethyl Ester Citrate | 1.5 | g |
| Ruteacarpine | 50 | mg |
| Cinnamon extract | 200 | mg |
| L-Arginine AKG | 1.5 | g |
| Glycocyamine | 1.0 | g |

1. A dietary supplement comprising an ester salt of creatine of the following structure, 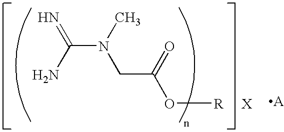
wherein R— contains alkyl and aryl groups or combinations thereof between 1 to 35 carbon atoms; n- represents 1 to 6 creatine; X— represents 1 to 6; and A- represents an organic acid with 1 to 6 carboxylic radicals.

2. A process of preparing a creatine ester salts comprising the following structure, 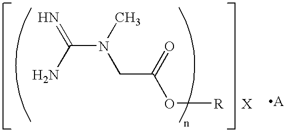
wherein R— contains alkyl and aryl groups or combinations thereof between 1 to 35 carbon atoms; n- represents 1 to 6 creatine; X— represents 1 to 6; and A- represents an organic acid with 1 to 6 carboxylic radicals; and
wherein the creatine ester salt is prepared at a temperature from −60° C. to 300° C. in a water solution or organic solution, or without any solvent.

3. The process according to claim 2 where, R is straight or branched, saturated or unsaturated.

4. The process according to claim 2 where, R further includes 1 to 6 hydroxy radicals.

5. The process according to claim 2 where R further includes substituent radicals selected from the group consisting of keto, halide, and amine.

6. The process according to claim 2 where, 1 to 6 carbon atoms in the 1 to 35 carbon atoms in R are replaced by a nitrogen, an oxygen, a sulphur or a phosphor atom.

7. (canceled)

8. (canceled)

9. A method for enhancing performance, muscle size or strength, comprising administering the dietary supplement according to claim 1.

10. A method according to claim 9, wherein from about 10 mg to about 20000 mg of the dietary supplement according to claim 1 is administered on a routine basis.

11. The process according to claim 2 wherein the R is selected from the group consisting of methyl, ethyl, propyl, isopropyl, butyl, isobutyl, tert-butyl glyceryl, and benzyl.

12. The process according to claim 2 wherein the A is selected from the group consisting of malic acid, maleic acid, fumaric acid, pyruvic acid, citric acid, tartaric acid, alpha-ketoglutaric acid and ascorbic acid.

13. The dietary supplement according to claim 1 wherein R is straight or branched, saturated or unsaturated.

14. The dietary supplement according to claim 1 wherein R has 1 to 6 hydroxy radicals.

15. The dietary supplement according to claim 1 wherein R has a substituent radical selected from the group consisting of keto, halide, and amine.

16. The dietary supplement according to claim 1 wherein 1 to 6 carbon atoms in the 1 to 35 carbon atoms in R are replaced by a nitrogen, an oxygen, a sulphur, or a phosphor atom.

17. The dietary supplement according to claim 1 wherein the R is selected from the group consisting of methyl, ethyl, propyl, isopropyl, butyl, isobutyl, tert-butyl, glyceryl, and benzyl.

18. The dietary supplement according to claim 1 wherein the A is selected from the group consisting of malic acid, maleic acid, fumaric acid, pyruvic acid, citric acid, tartaric acid, alpha-ketoglutaric acid and ascorbic acid.

19. The dietary supplement according to claim 1 wherein R— represents ethyl; n- represents 1; X— represents 1; and A- represents malic acid.

20. The method according to claim 9, wherein from about 1 g to about 20 g of the dietary supplement according to claim 1 is administered on a routine basis.
